(19) United States
(12) Patent Application Publication
(10) Pub. No.: US 2022/0325247 A1
(43) Pub. Date: Oct. 13, 2022

(54) METHOD FOR PROMOTING GROWTH OF PARIETAL DECIDUAL BASALIS-MESENCHYMAL STEM CELLS (PDB-MSCs)

(71) Applicant: GUANGDONG VITALIFE BIOTECHNOLOGY CO., LTD, Foshan (CN)

(72) Inventors: XIAOCUI LIU, Foshan (CN); YANYAO MENG, Foshan (CN); SHUYAN TANG, Foshan (CN); JUNRONG XU, Foshan (CN); SMEEN YA, Foshan (CN); JINGLI YANG, Foshan (CN); JINHUI WANG, Foshan (CN)

(21) Appl. No.: 17/720,279

(22) Filed: Apr. 13, 2022

(30) Foreign Application Priority Data
Apr. 13, 2021 (CN) 202110392687.8

(51) Int. Cl. C12N 5/0775; C12N 5/00

(52) U.S. Cl. CPC C12N 5/0668; C12N 5/0081; C12N 2502/025; C12N 2500/02; C12N 2500/90

(57) ABSTRACT

The present disclosure provides a method for promoting the growth of parietal decidual basalis-mesenchymal stem cells (PDB-MSCs), which comprises promoting the growth of parietal decidual basalis-mesenchymal stem cells (PDB-MSCs) via a human umbilical cord-mesenchymal stem cell (UC-MSC)-derived exosome. In the present disclosure, after the PDB-MSCs are co-cultivated with the human UC-MSC-derived exosome, the PDB-MSCs show strong cell proliferation ability, prominent cell shape, and desirable cell viability. That is, the human UC-MSC-derived exosome of the present disclosure can improve a quality of PDB-MSCs and effectively improve the ability of PDB-MSCs to secrete vascular endothelial growth factor (VEGF) and stem cell factor (SCF), so as to solve the problem that PDB-MSCs show decreased proliferation ability and poor cell viability after multiple passages, which effectively facilitates the large-scale cultivation and clinical practice of PDB-MSCs.

### CROSS REFERENCE TO RELATED APPLICATION

The present application claims the benefit of Chinese Patent Application No. 202110392687.8 filed on Apr. 13, 2021, the contents of which are incorporated herein by reference in their entirety.

### TECHNICAL FIELD

The present disclosure relates to the field of biotechnologies, and a method for promoting the growth of PDB-MSCs, and in particular to a method of promoting the growth of parietal decidual basalis-mesenchymal stem cells (PDB-MSCs) via exosomes.

### BACKGROUND

Parietal decidual basalis-mesenchymal stem cells (PDB-MSCs) have the common characteristics of mesenchymal stem cells (MSCs) such as multi-directional differentiation potential and low immunogenicity. Compared with MSCs derived from other sources, PDB-MSCs derived from a parent body show better compatibility with the parent body, and more uniform purity. PDB-MSCs derived from a parent body also show stronger adipogenic differentiation ability than MSCs derived from other tissues in a perinatal period. In addition, it is reported that PDB-MSCs secrete a larger amount of vascular endothelial growth factor (VEGF) and stem cell factor (SCF) than umbilical cord-mesenchymal stem cells (UC-MSCs), and thus have functions such as promoting angiogenesis and cell division, indicating that PDB-MSCs play an important role in tissue damage repair. PDB-MSCs have promising prospects in the clinical application of stem cells.

However, at present, the application of PDB-MSCs still faces some problems: PDB-MSCs with high purity can be isolated with the existing methods and technologies, but after passage, PDB-MSCs are prone to senescence, cell flattening, slow or stopped proliferation, differentiation loss, and the like. Therefore, a new method or technology is required to effectively maintain the morphology of PDB-MSCs, prevent cell senescence, and maintain the proliferation viability and differentiation ability of PDB-MSCs after multiple passages.

Exosomes are lipid-embolized molecular vesicles with a diameter of 30 nm to 100 nm. Almost all types of cells, including tumor cells, can produce and release exosomes. Exosomes include cell-specifically expressed nucleic acids, lipids, proteins, and the like, and can transfer biomolecules to target cells in contact with the external environment through cell membrane fusion, which can promote the proliferation of target cells.

Chinese Patent CN105349487 discloses a method for promoting the proliferation of human bone marrow-mesenchymal stem cells (BM-MSCs) based on an exosome, where an exosome secreted by the first or second generation of human UC-MSCs is used to promote the proliferation of human BM-MSCs, and a content and source of the exosome are greatly restricted. In addition, the culture medium for exosome-promoting human bone marrow mesenchymal stem cells contains 5% UltraGRO by mass and α-MEM basal medium containing 0.032% medical heparin sodium by mass. The formula of this medium is complicated and it is difficult to reproduce the experimental effect. Secondly, when the experiments to promote the proliferation of mesenchymal stem cells are carried out for scale-up culture, the operation is cumbersome. In addition, MSCs from different tissues may show some differences in properties and functions, for example, PDB-MSCs and BM-MSCs are not exactly the same in apoptosis tolerance and immunoreactivity, which causes the two to behave differently in cell migration, tissue repair, and the like.

### SUMMARY

A technical problem to be solved by the present disclosure is to provide a method for promoting the growth of PDB-MSCs, so as to effectively promote the proliferation of PDB-MSCs, shorten the cell doubling time, increase a proportion of cells at an S phase of a cell cycle, improve a quality of PDB-MSCs, and effectively improve the ability of PDB-MSCs to secrete VEGF and SCF, which is beneficial to the large-scale cultivation and clinical practice of PDB-MSCs.

In order to solve the above technical problem, the present disclosure provides a method for promoting the growth of PDB-MSCs via a human UC-MSC-derived exosome.

As an improvement of the above solution, the human UC-MSC-derived exosome may be secreted by a P5 generation of human UC-MSCs.

As an improvement of the above solution, a preparation method of the human UC-MSC-derived exosome may include:

S1. inoculating a P4 generation of human UC-MSCs into a culture flask at a concentration of 9,000 to 12,000 cells/cm², adding 20 ml to 30 ml of a serum-free proliferation medium, and cultivating;

S2. when confluency of the P4 generation of human UC-MSCs reaches 90%, collecting a culture supernatant to obtain a first supernatant;

S3. centrifuging the first supernatant, and collecting a resulting supernatant to obtain a second supernatant; and

S4. filtering the second supernatant to obtain a first filter residue, centrifuging the first filter residue, and collecting a precipitate at a bottom; and resuspending the precipitate with 0.8 ml to 1.5 ml of phosphate buffered saline (PBS), and filtering to obtain a second filter residue, which is the human UC-MSC-derived exosome.

As an improvement of the above solution, a preparation method of the P4 generation of human UC-MSCs may include:

S11. washing an umbilical cord tissue with a tissue protection solution to remove blood on a surface, removing epidermal and vascular tissues, and taking out Wharton's jelly; and washing the Wharton's jelly, cutting the Wharton's jelly into pieces of 1 to 2 mm³, and inoculating and cultivating according to a tissue adhesion method; and

S12. when primary cells grow to confluency of 70%, subculturing, and repeating the subculturing until the P4 generation is obtained.

As an improvement of the above solution, in S12, after a generation of cells grow to confluency of greater than 80%, the cells may be washed at least twice with PBS and then digested with trypsin for 4 min to 7 min, a medium may be added to terminate the digestion, a resulting mixture may be filtered and centrifuged, and a resulting precipitate may be resuspended with a cell medium and subcultured, which is repeated until the P4 generation is obtained.

As an improvement of the above solution, the trypsin may be a 20% to 30% Tryple-EDTA enzyme.

As an improvement of the above solution, in S11, the tissue protection solution may be prepared from 0.5 ml to 3 ml of normal saline (NS), 20 μg to 30 μg of gentamicin sulfate, and 3 μg to 6 μg of amphotericin B.

In some embodiments, the present disclosure also provides a method for promoting the growth of PDB-MSCs, including the steps of:

A. adding the human UC-MSC-derived exosome described above to an MSC serum-free medium at a concentration of 10 to 30 μg/ml to obtain an exosome-containing cell medium;

B. inoculating the PDB-MSCs in a cell culture plate at a concentration of 1.5×10³ to 2.5×10³ cells/well, and adding the exosome-containing cell medium; and

C. incubating the cell culture plate in an incubator, with a temperature of 35° C. to 40° C., a CO₂ concentration of 4% to 7%, and an O₂ concentration of 1% to 3%.

As an improvement of the above solution, in step B, a P12 generation of PDB-MSCs may be inoculated in the cell culture plate.

As an improvement of the above solution, in step A, the human UC-MSC-derived exosome may be added to the MSC serum-free medium at a concentration of 20 μg/ml.

The implementation of the present disclosure has the following beneficial effects:

1. The method for promoting the growth of PDB-MSCs via a human UC-MSC-derived exosome provided by the present disclosure has not been disclosed in any literatures or patents.

2. In the present disclosure, a P5 generation of human UC-MSCs are used to extract the exosome, which broadens extraction conditions of the exosome to obtain a larger amount of the exosome; and a quality of the exosome of the P5 generation of human UC-MSCs is similar to a quality of an exosome of a P2 generation of human UC-MSCs.

3. In the present disclosure, human UC-MSCs are extracted using a tissue protection solution containing 0.5 ml to 3 ml of NS, 20 μg to 30 μg of gentamicin sulfate, and 3 μg to 6 μg of amphotericin B, which can not only improve the extraction efficiency of human UC-MSCs but also the quality of extracted human UC-MSCs. Moreover, here 20% to 30% Tryple-EDTA enzyme was used to digest human UC-MSCs, which further ensure the quality of human UC-MSC derived exosome during subculturing.

4. The method for extracting the exosome from human UC-MSCs in the present disclosure is efficient and convenient, has a high extraction yield, and leads to a relatively high purity.

5. The human UC-MSC-derived exosome of the present disclosure can effectively promote the proliferation of PDB-MSCs, shorten the cell doubling time, and increase a proportion of cells at an S phase of a cell cycle.

6. In the present disclosure, after the PDB-MSCs are co-cultivated with the human UC-MSC-derived exosome, the PDB-MSCs show strong cell proliferation ability, prominent cell shape, and desirable cell viability. That is, the human UC-MSC-derived exosome of the present disclosure can improve a quality of PDB-MSCs and effectively improve the ability of PDB-MSCs to secrete VEGF and SCF, so as to solve the problem that PDB-MSCs show decreased proliferation ability and poor cell viability after multiple passages, which effectively facilitates the large-scale cultivation and clinical practice of PDB-MSCs.

### BRIEF DESCRIPTION OF THE DRAWINGS

FIG. 1 shows the morphology of the P5 generation of human UC-MSCs in Example 1 of the present disclosure;

FIG. 2 is a scanning electron microscopy (SEM) image of the human UC-MSC-derived exosome in Example 1 of the present disclosure;

FIG. 3 shows the morphology of PDB-MSCs cultivated with the human UC-MSC-derived exosome at different concentrations in Example 2 of the present disclosure;

FIG. 4 shows cell growth curves of PDB-MSCs cultivated with the human UC-MSC-derived exosome at different concentrations in Example 2 of the present disclosure;

FIG. 5 is a schematic diagram illustrating a proportion of cells at each stage of the cell cycle in PDB-MSCs cultivated with the human UC-MSC-derived exosome at different concentrations in Example 3 of the present disclosure;

FIG. 6 is a histogram illustrating the secretion of VEGF after a P12 generation of PDB-MSCs grow to the logarithmic growth phase in the cell media with different concentrations of exosome in Example 4 of the present disclosure (PDB-MSCs are cultivated in media with different concentrations of exosome); and

FIG. 7 is a histogram illustrating the secretion of SCF after a P12 generation of PDB-MSCs grow to the logarithmic growth phase in the cell media with different concentrations of exosome in Example 4 of the present disclosure (PDB-MSCs are cultivated in media with different concentrations of exosome).

### DETAILED DESCRIPTION

In order to make the objectives, technical solutions, and advantages of the present disclosure more clear, the present disclosure will be further described in detail below with reference to the accompanying drawings. It should be noted that orientation terms such as “upper”, “lower”, “left”, “right”, “front”, “rear”, “inner”, and “outer” that appear or are about to appear in the present disclosure are only based on the accompanying drawings of the present disclosure, and do not specifically limit the present disclosure.

### Example 1

A preparation method of a human UC-MSC-derived exosome was provided, including the following steps:

S11. A placental tissue delivered by caesarean section at term was collected in a hospital, and then transported to a laboratory in a refrigerated and sterile environment at 4° C. Before the collection, an informed consent form was signed by a customer. During the transportation, a tissue protection solution was used to protect the biological activity of the placental tissue and prevent the placental tissue from bacterial and fungal contamination. The tissue protection solution was prepared from 1 ml of NS, 25 μg of gentamicin sulfate, and 5 μg of amphotericin B.

S12. In the laboratory, the umbilical cord tissue was washed with the tissue protection solution to remove blood on a surface, epidermal and vascular tissues were removed, and Wharton's jelly was taken out, washed, cut into pieces of 1 to 2 mm³, and inoculated in a T75 culture flask and cultivated with a serum-free selective medium of well-known components according to a tissue adhesion method.

S13. After growing to confluency of 70% on day 14 of cultivation, primary cells were subcultured; and according to a conventional cell cultivation operation, the subculturing was conducted until a P4 generation was obtained (Subculturing: after a generation of cells grew to confluency of greater than 80%, the cells were washed twice with PBS and then digested with trypsin for 5 min, a medium was added to terminate the digestion, a resulting mixture was filtered and centrifuged, and a resulting precipitate was resuspended with a cell medium, counted to calculate a survival rate, and subcultured. The trypsin was a 25% Tryple-EDTA enzyme).

S14. UC-MSCs with well growth conditions in the P4 generation were collected and inoculated in a Corning T175 culture flask at a density of 10,000 cells/cm², 25 ml of a serum-free proliferation medium was added, and then the cells were cultivated normally.

S15. When the cells grew to confluency of 90%, a culture supernatant was collected (a culture supernatant of human UC-MSCs in the P5 generation, as shown in FIG. 1) to obtain a first supernatant, which would be used to extract an exosome; and the MSCs were subcultured normally, and a cultivation generation was determined according to a cell growth state.

S16. The collected first supernatant was centrifuged at 3,000 rpm for 20 min to remove cell debris, and a resulting supernatant was collected to obtain a second supernatant.

S17. The second supernatant was filtered with a 0.22 μL filter membrane and centrifuged at 120,000 rpm for 120 min, a resulting supernatant was discarded, and a resulting precipitate (which was the human UC-MSC-derived exosome) was resuspended with 1 ml of PBS and filtered with a 0.22 μL filter membrane, which was temporarily stored at 4° C. and long-term stored at −80° C.

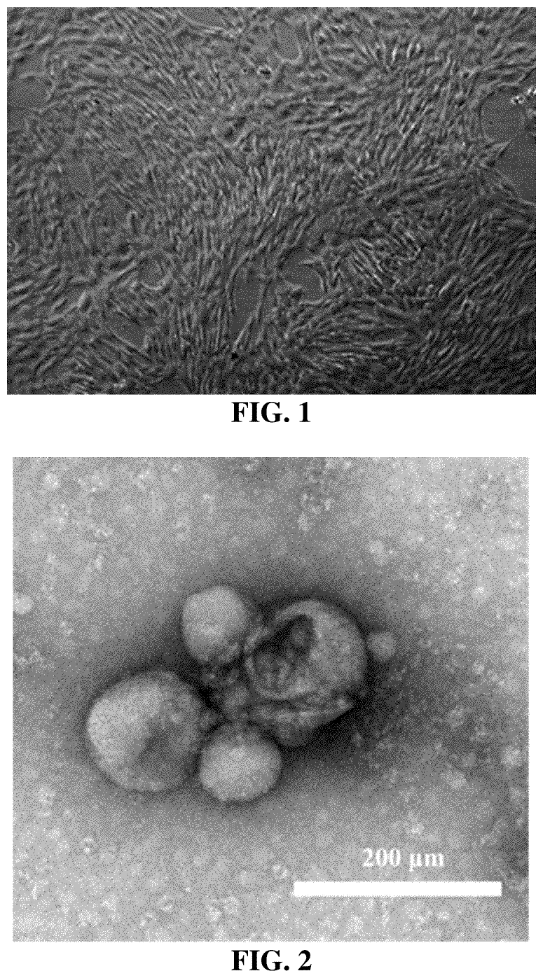

An SEM image of the human UC-MSC-derived exosome is shown in FIG. 2, and it can be seen that the human UC-MSC-derived exosome is in a shape of a saucer or a hemisphere with a concave side.

### Example 2

A method for promoting the proliferation of PDB-MSCs was provided, including the following steps:

S21. Preparation of cell media with different concentrations of exosome: the human UC-MSC-derived exosome prepared in Example 1 was added at different concentrations (0 μg/ml, 10 μg/ml, 20 μg/ml, and 30 μg/ml) to different MSC serum-free media (Youkang Hengye Biotechnology (Beijing) Co., Ltd., Item NO.: NC0103).

S22. A P12 generation of PDB-MSCs were inoculated in a 96-well plate at a concentration of 2×10³ cells/well, and the exosome-containing cell media obtained in S21 were added separately.

S23. The cells were cultivated in an incubator with saturated humidity at 37° C., 5% CO₂, and 1% O₂.

Each experiment was repeated at least three times.

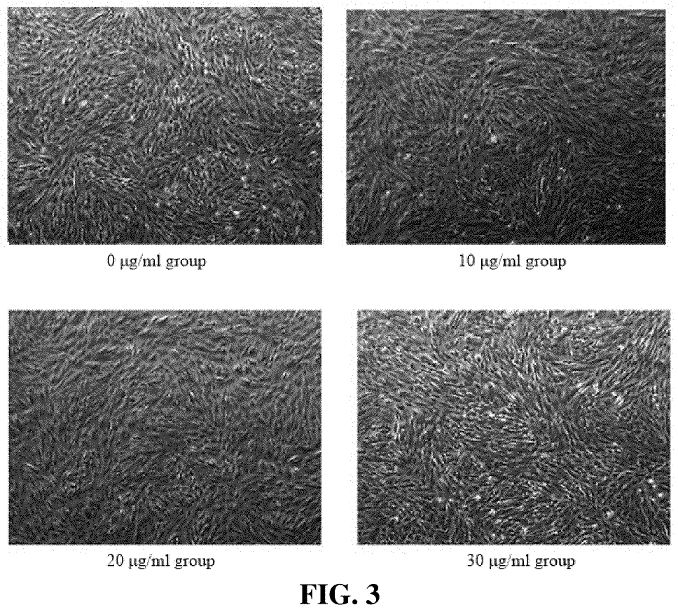

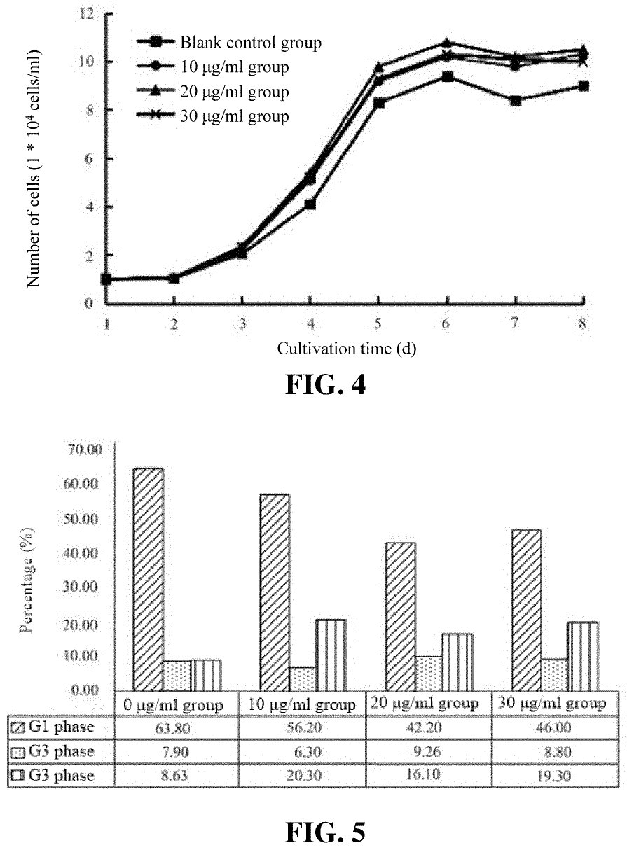

PDB-MSCs were collected every 24 h and counted. FIG. 3 shows the morphology of PDB-MSCs cultivated with the human UC-MSC-derived exosome at different concentrations in Example 2. FIG. 4 shows cell growth curves of PDB-MSCs cultivated with the human UC-MSC-derived exosome at different concentrations in Example 2.

It can be seen from FIG. 3 and FIG. 4 that the growth of PDB-MSCs is accelerated when the PDB-MSCs are cultivated in the cell media with different concentrations of exosome.

### Example 3

A method for promoting the proliferation of PDB-MSCs was provided, including the following steps:

S21. Preparation of cell media with different concentrations of exosome: the human UC-MSC-derived exosome prepared in Example 1 was added at different concentrations (0 μg/ml, 10 μg/ml, 20 μg/ml, and 30 μg/ml) to different MSC serum-free media (Youkang Hengye Biotechnology (Beijing) Co., Ltd., Item NO.: NC0103).

S22. A P12 generation of PDB-MSCs were inoculated in a 96-well plate at a concentration of 4×10⁵ cells/well, and the exosome-containing cell media obtained in S21 were added separately.

S23. The cells were cultivated in an incubator with saturated humidity at 37° C., 5% CO₂, and 1% O₂.

Each experiment was repeated at least three times.

PDB-MSCs were collected after 48 h of cultivation and reacted with a cell cycle detection kit (Solarbio) for 30 min, and then G1, G2, and S phases of the cell cycle were tested by a flow cytometer (Beckman). FIG. 5 shows the flow cytometry results of cell cycle of PDB-MSCs cultivated with the human UC-MSC-derived exosome at different concentrations in Example 3.

It can be seen from FIG. 5 that a proportion of cells at the S phase of the cell cycle increases when PDB-MSCs are cultivated in the cell media with different concentrations of exosome, and a proportion of cells at the S phase in the group with an exosome content of 20 μg/ml is higher than that in other groups, indicating that the co-cultivation of PDB-MSCs with the human UC-MSC-derived exosome added to a medium can accelerate the DNA replication and proliferation of PDB-MSCs.

### Example 4

A method for promoting the proliferation of PDB-MSCs was provided, including the following steps:

S21. Preparation of cell media with different concentrations of exosome: the human UC-MSC-derived exosome prepared in Example 1 was added at different concentrations (0 μg/ml, 10 μg/ml, 20 μg/ml, and 30 μg/ml) to different MSC serum-free media (Youkang Hengye Biotechnology (Beijing) Co., Ltd., Item NO.: NC0103).

S22. A P12 generation of PDB-MSCs were inoculated in a 96-well plate at a concentration of 2.5×10⁵ cells/well, and the exosome-containing cell media obtained in S21 were added separately.

S23. The cells were cultivated in an incubator with saturated humidity at 37° C., 5% CO₂, and 1% O₂.

Each experiment was repeated at least three times.

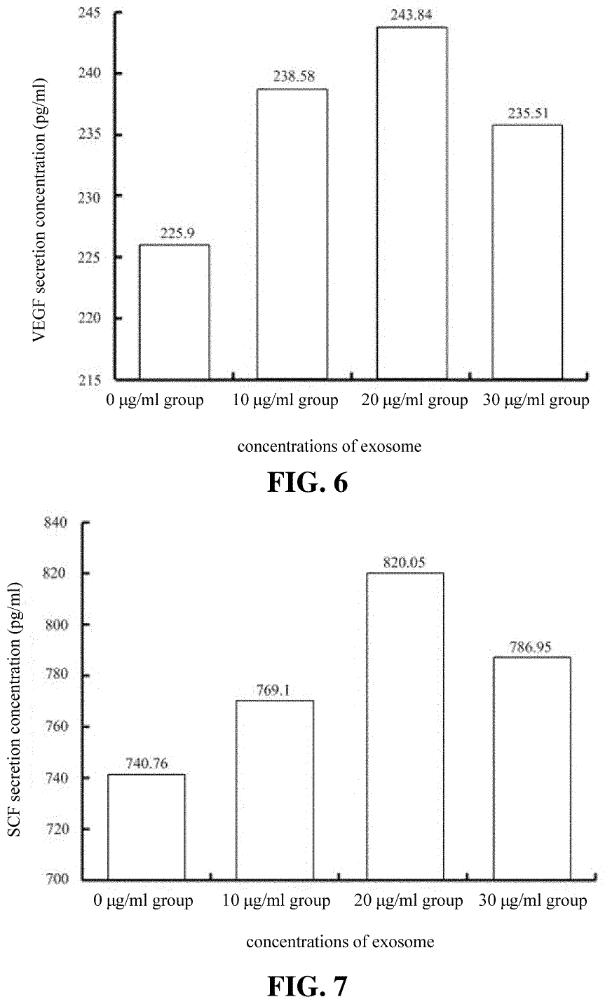

When a P12 generation of PDB-MSCs grew to the logarithmic growth phase, 1 ml of a culture supernatant was collected and centrifuged at 3,000 rpm for 20 min to obtain a cell supernatant, and the VEGF and SCF ELISA kits (Enzyme-linked Biotechnology Co., Ltd.) were respectively used to detect VEGF and SCF contents in the cell supernatant. FIG. 6 is a histogram illustrating the secretion of VEGF after the P12 generation of PDB-MSCs grow to the logarithmic growth phase in the cell media with different concentrations of exosome in Example 4; and FIG. 7 is a histogram illustrating the secretion of SCF after the P12 generation of PDB-MSCs grow to the logarithmic growth phase in the cell media with different concentrations of exosome in Example 4.

It can be seen from FIG. 6 and FIG. 7 that when the PDB-MSCs are cultivated in the cell media with different concentrations of exosome, the secretion of both VEGF and SCF in PDB-MSCs increases, indicating that the addition of the human UC-MSC-derived exosome can promote the secretion of both VEGF and SCF in PDB-MSCs.

The above are only preferred examples of the present disclosure, and are not intended to limit the claimed scope of the present disclosure. Therefore, equivalent changes made according to the claims of the present disclosure are still within the scope of the present disclosure.

1. A method for promoting the growth of parietal decidual basalis-mesenchymal stem cells (PDB-MSCs), wherein the method comprises
promoting the growth of parietal decidual basalis-mesenchymal stem cells (PDB-MSCs) via a human umbilical cord-mesenchymal stem cell (UC-MSC)-derived exosome.

2. The method according to claim 1, wherein the human UC-MSC-derived exosome is secreted by a P5 generation of human UC-MSCs.

3. The method according to claim 2, wherein a preparation method of the human UC-MSC-derived exosome comprises:
S1. inoculating a P4 generation of human UC-MSCs into a culture flask at a concentration of 9,000 to 12,000 cells/cm², adding 20 ml to 30 ml of a serum-free proliferation medium, and cultivating;
S2. when confluency of the P4 generation of human UC-MSCs reaches 90%, collecting a culture supernatant to obtain a first supernatant;
S3. centrifuging the first supernatant, and collecting a resulting supernatant to obtain a second supernatant; and
S4. filtering the second supernatant to obtain a first filter residue, centrifuging the first filter residue, and collecting a precipitate at a bottom; and resuspending the precipitate with 0.8 ml to 1.5 ml of phosphate buffered saline (PBS), and filtering to obtain a second filter residue, which is the human UC-MSC-derived exosome.

4. The method according to claim 3, wherein a preparation method of the P4 generation of human UC-MSCs comprises:
S11. washing an umbilical cord tissue with a tissue protection solution to remove blood on a surface, removing epidermal and vascular tissues, and taking out Wharton's jelly; and washing the Wharton's jelly, cutting the Wharton's jelly into pieces of 1 to 2 mm³, and inoculating and cultivating according to a tissue adhesion method; and
S12. when primary cells grow to confluency of 70%, subculturing, and repeating the subculturing until the P4 generation is obtained.

5. The method according to claim 4, wherein in S12, after a generation of cells grow to confluency of greater than 80%, the cells are washed at least twice with PBS and then digested with trypsin for 4 min to 7 min, a medium is added to terminate the digestion, a resulting mixture is filtered and centrifuged, and a resulting precipitate is resuspended with a cell medium and subcultured, which is repeated until the P4 generation is obtained.

6. The method according to claim 5, wherein the trypsin is a 20% to 30% Tryple-EDTA enzyme.

7. The method according to claim 4, wherein in S11, the tissue protection solution is prepared from 0.5 ml to 3 ml of normal saline (NS), 20 μg to 30 μg of gentamicin sulfate, and 3 μg to 6 μg of amphotericin B.

8. A method for promoting the growth of PDB-MSCs, comprising the steps of:
A. adding the human UC-MSC-derived exosome according to claim 1 to an MSC serum-free medium at a concentration of 10 to 30 μg/ml to obtain an exosome-containing cell medium;
B. inoculating the PDB-MSCs in a cell culture plate at a concentration of 1.5×10³ to 2.5×10³ cells/well, and adding the exosome-containing cell medium; and
C. incubating the cell culture plate in an incubator, with a temperature of 35° C. to 40° C., a CO₂ concentration of 4% to 7%, and an O₂ concentration of 1% to 3%.

9. A method for promoting the growth of PDB-MSCs, comprising the steps of:
A. adding the human UC-MSC-derived exosome according to claim 2 to an MSC serum-free medium at a concentration of 10 to 30 μg/ml to obtain an exosome-containing cell medium;
B. inoculating the PDB-MSCs in a cell culture plate at a concentration of 1.5×10³ to 2.5×10³ cells/well, and adding the exosome-containing cell medium; and
C. incubating the cell culture plate in an incubator, with a temperature of 35° C. to 40° C., a CO₂ concentration of 4% to 7%, and an O₂ concentration of 1% to 3%.

10. A method for promoting the growth of PDB-MSCs, comprising the steps of:
A. adding the human UC-MSC-derived exosome according to claim 3 to an MSC serum-free medium at a concentration of 10 to 30 μg/ml to obtain an exosome-containing cell medium;
B. inoculating the PDB-MSCs in a cell culture plate at a concentration of 1.5×10³ to 2.5×10³ cells/well, and adding the exosome-containing cell medium; and
C. incubating the cell culture plate in an incubator, with a temperature of 35° C. to 40° C., a CO₂ concentration of 4% to 7%, and an O₂ concentration of 1% to 3%.

11. A method for promoting the growth of PDB-MSCs, comprising the steps of:
A. adding the human UC-MSC-derived exosome according to claim 4 to an MSC serum-free medium at a concentration of 10 to 30 μg/ml to obtain an exosome-containing cell medium;
B. inoculating the PDB-MSCs in a cell culture plate at a concentration of 1.5×10³ to 2.5×10³ cells/well, and adding the exosome-containing cell medium; and
C. incubating the cell culture plate in an incubator, with a temperature of 35° C. to 40° C., a CO₂ concentration of 4% to 7%, and an O₂ concentration of 1% to 3%.

12. A method for promoting the growth of PDB-MSCs, comprising the steps of:
A. adding the human UC-MSC-derived exosome according to claim 5 to an MSC serum-free medium at a concentration of 10 to 30 μg/ml to obtain an exosome-containing cell medium;
B. inoculating the PDB-MSCs in a cell culture plate at a concentration of 1.5×10³ to 2.5×10³ cells/well, and adding the exosome-containing cell medium; and
C. incubating the cell culture plate in an incubator, with a temperature of 35° C. to 40° C., a CO₂ concentration of 4% to 7%, and an O₂ concentration of 1% to 3%.

13. A method for promoting the growth of PDB-MSCs, comprising the steps of:
A. adding the human UC-MSC-derived exosome according to claim 6 to an MSC serum-free medium at a concentration of 10 to 30 μg/ml to obtain an exosome-containing cell medium;
B. inoculating the PDB-MSCs in a cell culture plate at a concentration of 1.5×10³ to 2.5×10³ cells/well, and adding the exosome-containing cell medium; and
C. incubating the cell culture plate in an incubator, with a temperature of 35° C. to 40° C., a CO₂ concentration of 4% to 7%, and an O₂ concentration of 1% to 3%.

14. A method for promoting the growth of PDB-MSCs, comprising the steps of:
A. adding the human UC-MSC-derived exosome according to claim 7 to an MSC serum-free medium at a concentration of 10 to 30 μg/ml to obtain an exosome-containing cell medium;
B. inoculating the PDB-MSCs in a cell culture plate at a concentration of 1.5×10³ to 2.5×10³ cells/well, and adding the exosome-containing cell medium; and
C. incubating the cell culture plate in an incubator, with a temperature of 35° C. to 40° C., a CO₂ concentration of 4% to 7%, and an O₂ concentration of 1% to 3%.

15. The method for promoting the growth of PDB-MSCs according to claim 8, wherein in step B, a P12 generation of PDB-MSCs are inoculated in the cell culture plate.

16. The method for promoting the growth of PDB-MSCs according to claim 9, wherein in step B, a P12 generation of PDB-MSCs are inoculated in the cell culture plate.

17. The method for promoting the growth of PDB-MSCs according to claim 10, wherein in step B, a P12 generation of PDB-MSCs are inoculated in the cell culture plate.

18. The method for promoting the growth of PDB-MSCs according to claim 8, wherein in step A, the human UC-MSC-derived exosome is added to the MSC serum-free medium at a concentration of 20 μg/ml.

19. The method for promoting the growth of PDB-MSCs according to claim 9, wherein in step A, the human UC-MSC-derived exosome is added to the MSC serum-free medium at a concentration of 20 μg/ml.

20. The method for promoting the growth of PDB-MSCs according to claim 10, wherein in step A, the human UC-MSC-derived exosome is added to the MSC serum-free medium at a concentration of 20 μg/ml.
